(19) United States
(12) Patent Application Publication
(10) Pub. No.: US 2005/0015840 A1
(43) Pub. Date: Jan. 27, 2005

(54) Forearm sun protection garment

(76) Inventor: James M. Biggerstaff, Grove, OK (US)

Correspondence Address: JAMES R. HEAD, 28926 SOUTH 593 RD., GROVE, OK, 74344, US

(21) Appl. No.: 10/627,133

(22) Filed: Jul. 25, 2003

(51) Int. Cl. A61F 9/00; A41D 13/08

(52) U.S. Cl. 2/16

(57) ABSTRACT

An apparatus for protecting the exposed top portion of human arms, one or both, from the sun's rays. A piece of flexible fabric, preferably with an SPF of at least 15, which covers at most the top half of the arm is supported by spaced first and second elastic bands which encircle the Ulna or wrist and the elbow, including a portion of the Humerus, or portion of the upper part of the arm, leaving the arm there between to receive the free flow of cooling air around and under the flexible fabric portion. In one embodiment, that portion of the elastic band adjacent and touching the elbow bone is a softer expandable portion to allow non binding movement thereof. Another aspect of the invention is where the outside of the flexible fabric is adaptable to contain advertising, such as, but not limited to, goods, services, school or university colors, emblems or expressions, and the like.

### FIELD OF THE INVENTION

The present invention relates to special solar protection garments. and, more particularly, to devices for prevention of burns or other damaging skin diseases to the forearms. The device is particularly adaptable for use by individuals who work and play in the sun.

### BACKGROUND OF THE INVENTION

Most who work in the open air usually wear short sleeves shirts leaving the forearms exposed. One of the basic problems confronting those who work, or play with their forearms exposed, is sunburn or the possibility of other skin diseases caused by the sun's rays. On the other hand it is also desirable to keep the arms as cool as possible.

Within just five minutes on a sunny summer day one's skin may absorb enough UV radiation to develop a minor sunburn. Skin cells may suffer injury that can not be seen or felt. Multiply that over the years and damage from daily sun exposure may become major, including skin cancer and skin aging. In recent years, the public has become more aware of the effects of melanoma and the prevention and treatment thereof.

There are two types of ultraviolet rays—UVA (long wave solar rays of 320-400 nanometers) and UVB (short wave solar rays of 290-320 nanometers)—that can affect the skin even with small daily doses. The UVB rays are most abundant in the midday sun, usually from 10 a.m. to 4 p.m., when your shadow is shorter than a person, and are most associated with sunburn. The medical community, recommends full head-to-toe protection of at least SPF (Sun Protection Factor) of 15, and preferably 30+. SPF or UPF (Ultra Violet Protection Factor) is meant to be a guide of how much time you can spend in the sun compared to how long the skin takes to redden without protection. Liquid sun screens, although effective, are troublesome to use and are not always effective against harmful UVA radiation. It is known that most people don't apply the recommended doses of sun screen and/or don't like the mess. For more information refer to the Internet at <www.skincancer.org>.

Others have tried to seek protection of their arms by tubular sleeves that surround the arm and in some instances up to and including the shoulder. Some representative samples of such garments can be found in the following U.S. Pat. Nos.:

| | |
|---|---|
| 5,357,633 | Rael |
| 5,628,062 | Tseng |
| 5,974,586 | Reinoso |
| 6,539,550 | Flores |

However, these devices, although they teach sun protective concepts, are tubular in construction being designed to fully cover the arm and in the instance of the Rael, Flores and Reinoso to include the upper arm and shoulder. Such devices would not be comfortable in the heat of summer as there is no free flow of air around the exposed limbs. Other forms of devices to protect the arms and/or shoulders for different, or unexplained, reasons are found in the following U.S. Pat. Nos.:

| | |
|---|---|
| 1,141,656 | Rosenbaum, et al. |
| 1,157,341 | Tallerday |
| 5,511,241 | Ziegler |
| 5,734,992 | Ross |
| 6,405,381 | Bowman, Jr. |
| 6,449,772 | Donner |
| 6,472,590 | Kulik |

There are in the prior art devices for partially covering the arm as shown in the following U.S. Pat. Nos.:

| | |
|---|---|
| 794,294 | Gardner |
| 925,952 | Sacks |
| 5,056,157 | Pryor |

Gardner and Sacks describe devices for protecting the underside of sleeve covered arms such as during indoor deskwork.

The Pryor patent describes a device for protecting the upper forearm from solar radiation, but like the prior art utilizes a plurality of VELCRO straps to releasably secure the fabric. To permit free flow of air, as when the arm is outside the window of a vehicle, these straps must be loose fitting which could allow the device to move about the arm. To prevent that, the straps must then be tight around the arm, which then will restricting the free flow of air around the arm.

Although protecting the arm which projects outwardly of a vehicle is still possible, the modern day vehicle is usually air cooled with the windows closed. There are, however, many other situations where protection is needed. Construction laborers, service personnel, and even those who work and attend outdoor sports or other events could use protective devices.

It is therefore an object of the invention to provide a garment device for protecting one or both forearms of individuals from the harmful effects of the sun.

It is another object of the invention to provide a device that not only protects the forearm of individuals but is comfortable and cooler. The distinctive feature being a length of flexible sun blocking fabric that is loosely fitted over at least the top portion of the arm between two elastic bands, one at the wrist, the other on each side of and covering an area on each side of the elbow joint and bone.

Another object of the invention is the use of a medically acceptable fabric providing high SPF, UVA and UVB sun protection such as that sold under the trademark SOLUMBRA, a product of Sun Precautions, Inc. Other fabrics of Solar Protective Factory, Inc sold under the trademarks SOLARWEAVE and SUN RePel are inclusive of use. Such fabrics are advertised to provide 30+ SPF protection.

A further object of the invention is to provide a fabric covering that includes promotional advertising on the outside.

### SUMMARY OF THE INVENTION

In accordance with the present invention, there is provided an apparatus for protecting one or both of the exposed top portion of a human forearm below a sleeve worn in the upper arm from the sun's rays. A piece of flexible fabric, preferably, but not limited to, a sun blocking material of at least SPF of 15. The fabric covering at least or at most the top half of the arm is supported by spaced first and second elastic bands. The first band encircles the Ulna or wrist. The second encircling the elbow, with a portion around the forearm and a portion around the Humerus, or upper part of the arm at or below the sleeve, leaving that portion of the forearm beneath the flexible fabric to receive the unobstructed free flow of air around and under the flexible fabric portion. In one embodiment, that portion of the elastic band contiguous, i.e., adjacent and touching, the elbow bone contains a softer expandable portion to allow non binding movement thereof. The elastic band itself can be of an open weave to provide some air flow yet maintain high SPF.

Another aspect of the invention is where the outside of the flexible fabric may contain informational indicia, ornamental designs, or even advertising, such as, but not limited to, goods, services, school or university colors, emblems or expressions, and the like.

### BRIEF DESCRIPTION OF THE DRAWINGS

A complete understanding of the present invention may be obtained by reference to the accompanying drawings, when considered in conjunction with the subsequent, detailed description.

FIG. 1 is a perspective view of the garment of this invention.

FIG. 2 is a side elevation view of the garment of this invention as applied to one arm.

FIG. 3 is a cross-sectional view taken along the line 3-3 of FIG. 2.

FIG. 4 is a partial front view of an individual wearing the garment of this invention.

For purposes of clarity and brevity, like elements and components will bear the same designations and numbering throughout the FIGURES.

### Reference Numerals

20 The garment generally.
22 Front or wrist elastic band.
24 Rear elastic band that extends on both the forearm and the upper arm.
25 Portion of 24 that covers part of the forearm.
26 Special insert in the rear elastic band 24 covering the elbow bone.
28 Flexible fabric that covers the upper half of the arm between 22 and 24.
29 Unobstructed air space beneath the flexible fabric 28.
30 Forearm.
32 Hand.
34 Upper arm.
36 Shirt sleeve.
Decoration

### DETAILED DESCRIPTION OF THE PREFERRED EMBODIMENT

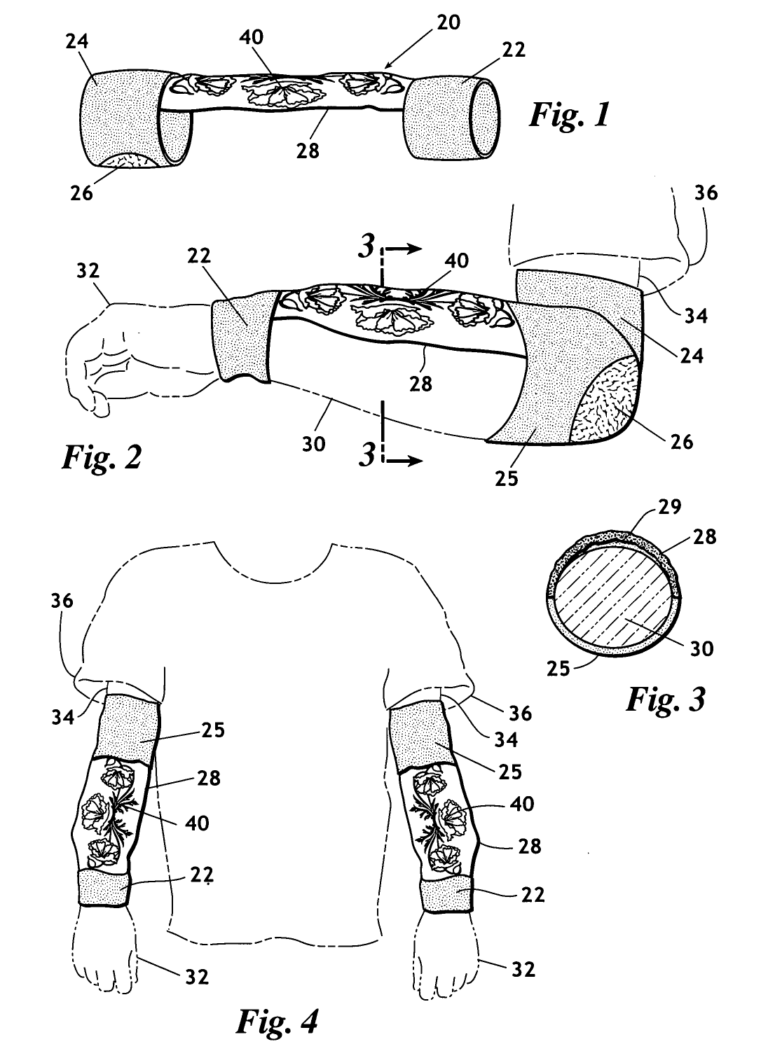

Referring now to FIG. 1 the garment of this invention, generally designated by the numeral 20, includes a front wrist elastic band 22 and a rear elastic band 24 that extends around the elbow from the forearm to the upper arm. In one embodiment a special ‘less elastic’ insert 26 covers the elbow to lessen the pressure off the bone and to permit movement. Between the front elastic band 22 and the rear elastic band 24 is a flexible fabric 28 that covers at least the top half of the persons forearm 30 as shown in FIG. 2.

Referring to FIGS. 2 and 3, the garment is shown with the flexible fabric 28 over the top half of the forearm 30 such that there is unobstructed air circulation thereabout. The fabric 28 is supported between a front elastic band 22, behind the hand 32, and a rear elastic band 24 that covers a portion of the forearm 23, the elbow, and a portion of the upper arm 34. In this view the special insert 26 is shown over the elbow. The elastic bands themselves can be an open weave or breathable material, yet sun protective.

FIG. 4 depicts a frontal view of the garments as they appear atop the arms with like numerals for like parts described heretofore.

As shown best in FIG. 4 the top 40 of the flexible fabric may be of color or design of the shirt 36 and/or contain decoration such as informational indicia, ornamental designs, or even advertising, such as, but not limited to, goods, services, school or university colors, emblems or expressions, and the like.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

1. A garment for protecting the exposed top portion of a human limb from the sun's rays, comprising:
a piece of flexible material covering the top portion of said limb, said material being supported by spaced first and second elastic bands, said first elastic band contiguous to said wrist, and said second elastic band contiguous to said elbow joint, said second elastic band including a softer expandable elastic portion essentially covering said elbow to allow non-binding movement, leaving that portion of said limb below said flexible material to receive unobstructed air circulation around said portion of limb yet protecting said top portion from said sun's rays.

2. The garment of claim 1 wherein said portion of the limb being protected is essentially a radius portion of the human forearm between a wrist and an elbow.

3. (Cancelled)

4. (Cancelled)

5. The garment of claim 1 wherein said flexible material is capable of providing at least 30 SPF sun block protection.

6. The garment of claim 5 wherein said flexible material includes an ornamental or informational design.

7. The garment of claim 1 wherein said elastic bands are of a breathable weave yet protective from the sun.

8. A garment for protecting the exposed top portion of a human forearm between a wrist and an elbow joint area from the sun's rays, comprising:
a piece of flexible material covering, at most, a top half of said top portion of said forearm, said material being supported by spaced first and second elastic bands, leaving that portion of said forearm below said flexible material to receive unobstructed air circulation around said portion of arm yet protecting said top portion from said sun's rays;
said first elastic band is contiguous to said wrist, and said second elastic band is contiguous to said elbow joint area;
second elastic band includes a softer expandable elastic portion essentially covering said elbow to allow non-binding movement.
said flexible material is capable of providing at least 15 SPF sun block protection; and wherein said flexible material includes an ornamental or informational design.

9. A garment for protecting from the sun's rays the exposed top portion of that portion of a human forearm, from a wrist to an elbow joint, comprising:
a single piece of strapless flexible material covering said top portion of said human forearm, said material being supported by spaced first and second elastic bands, said first elastic band contiguous to said wrist, and said second elastic band contiguous to said elbow joint, leaving that portion of said forearm below said strapless flexible material to receive unobstructed air circulation around said portion of said human forearm, yet protecting said top portion from said sun's rays.

10. The garment of claim 9 wherein said flexible material is capable of providing at least 30 SPF sun block protection.

11. The garment of claim 9 wherein said flexible material includes an ornamental or informational design.
